(19) United States
(12) Patent Application Publication
(10) Pub. No.: US 2004/0120915 A1
(43) Pub. Date: Jun. 24, 2004

(54) Multifunctional compositions for surface applications

(76) Inventors: Kaiyuan Yang, Cumming, GA (US); John Gavin MacDonald, Decatur, GA (US); Sohail Malik, Roswell, GA (US); Yanbin Huang, Roswell, GA (US)

Correspondence Address: KIMBERLY-CLARK WORLDWIDE, INC., 401 NORTH LAKE STREET, NEENAH, WI, 54956

(21) Appl. No.: 10/324,555

(22) Filed: Dec. 19, 2002

(51) Int. Cl. A61K 7/06; A61K 7/11; C11D 17/00; C11D 17/08

(52) U.S. Cl. 424/70.13; 424/70.14; 510/189; 510/404; 510/419

(57) ABSTRACT

The present invention relates to the preparation of surface cleaning and protection compositions. A typical embodiment of this invention may be a lotion, a soap, a cream, an aerosol, a gel, a medicinal lotion, a fabric cleanser, a furniture polish, an automobile polish, and a cleanser for other solid surfaces. The use of polyhederal oligomeric silsequioxane (POSS) allows greater flexibility in delivering active agents to a surface as well as for surface protection from irritants. The current invention discloses compositions for a variety of applications ranging from skin care, home hygiene, health care, entertainment, and children training/education. The compositions may provide molecular-level benefits, form breathable protection layers on a surface (e.g. skin), provide UV protection, provide foaming effects or allow users (consumers) to know when or if the cleaning is complete.

The present invention relates to the preparation of surface cleaning and protection compositions.

Technological innovations in surface cleaning and protection, particularly in skin care, are clearly trending to multifunctional products. Consumers want products that not only provide cosmetic benefits, but which also improve skin health and bring them comfort and enjoyment. Not only do they demand natural-based, anti-oxidant-rich products to reduce the appearance of fine lines and wrinkles, but they also want products to prevent premature aging and damage. The formulation industry thus continues to evolve better products with improved raw materials and better delivery of functional materials, while keeping the focus on aesthetics, beauty, and safety.

In trying to meet the customer demands for multifunctional products, more and more manufacturers are taking a “cocktail” approach: offering a variety of benefits in a single product by mixing all the necessary ingredients together in one product. One example of such a product is Proctor & Gamble's Olay with multiple vitamin ingredients for specially designed delivery systems. Another example is Clearance Time by Origins, an AHA (alpha hydroxyl acid) product for oily skin in which salicylic acid, seaweed extract, oat extract and silica microspheres are used together to absorb oil and give a matte finish.

Although there is nothing wrong with the “cocktail” approach, there are severe constraints in formulating such products. Firstly, it requires sophisticated formulation strategies to mix many ingredients together, a goal which is often difficult to achieve considering that the ingredients may have very different solubility and stability properties. Mixing multiple hydrophilic and hydrophobic ingredients together, for example, is not an easy task if these materials require different formulation temperatures or solvents. In addition, the stability of the finished product becomes a serious concern because emulsion systems with many ingredients are known to be fragile and often unstable for long-term storage.

Accordingly, there is a need for stable surface cleaning and protection compositions that can deliver multifunctional benefits. More particularly, there is a need to produce surface cleaning and protection formulations that contain one or more ingredients that are multifunctional. The term “multifunctional” means that a single ingredient can provide more than one benefits, as distinguished from the traditional “cocktail” approach. In addition, there is a need to produce surface cleaning and protection formulations that are not only multifunctional, but which are multifunctional at a molecular-level. There is, furthermore, a need to develop new formulation strategies for producing molecular-level, multifunctional products.

### SUMMARY OF THE INVENTION

In response to the discussed difficulties and problems encountered in the prior art, new compositions for a variety of applications ranging from skin care, home hygiene, health care, entertainment, and children training/education are disclosed. The use of polyhederal oligomeric silsequioxane (POSS) in the formulations allows greater flexibility in delivering active agents to a surface for cleaning as well as for surface protection from irritants. The compositions may provide molecular-level benefits such as forming breathable protection layers on a surface (e.g. skin), providing UV protection through the “hollow sphere” effect, providing foaming effects and allowing users to know when or if the cleaning is complete. Formulations benefiting from the inclusion of POSS compounds include lotions, creams, aerosols, gels, soaps, medicinal lotions, fabric cleansers, furniture polishes, automobile polishes, and cleansers for other solid surfaces.

### BRIEF DESCRIPTION OF THE DRAWINGS

FIG. 1 is a representative sketch of POSS hybrid having molecular-level functional ingredients. The R and X groups can be modified to be either hydrophobic or hydrophilic.

FIG. 2 is a three dimensional drawing of a multi-functional POSS compound with hydrophilic groups on the cage and hydrophilic OH group on the end of the arm. The cage area is suitable for small molecule such as oxygen to pass and also refract UV light.

FIG. 3 is a graph to show the UV light absorption of POSS compounds on a glass slide. The lowest line is the UV absorption of the glass slide alone. The middle line is the UV absorption after 5 minutes of drying and the upper line the UV absorption after 3 hours of drying.

FIG. 4 is a graph showing the enhancement of SPF numbers by a POSS compound in a sunscreen formulation that contains avobenzone and titanium dioxide. The Y-axis indicates absorbance and the X-axis indicates the wavelength from 250 to 400 nanometers. The square labeled lines are UV absorption spectrum for a POSS containing formulation while the triangle labeled lines are UV absorption spectrum for a POSS-free formulation.

FIG. 5 is a graph showing the SPF increase for a foaming sunscreen containing a POSS compound. The Y-axis indicates absorbance and the X-axis indicates the wavelength from 250 to 400 nanometers.

### DETAILED DESCRIPTION OF THE INVENTION

The present invention desirably provides molecular-level benefits to a variety of formulations such as lotions, creams, gels, soaps, detergents, etc. Molecular-level benefits means that the formulation can contact or interact with a variety of surfaces, one example being the skin, at a molecular level, and provide functional benefits at a molecular-level. Examples of such molecular-level benefits, particularly for skin care, include cleansing, shining, moisturizing, smoothing dryness, providing comfort, strengthening, UV protection, and so forth. In providing such desired molecular-level benefits, one or more of molecular-level functional ingredients is/are included in the formulation as described below.

A typical embodiment of this invention may be a lotion, a cream, an aerosol, a gel, a soap, a medicinal lotion, a fabric cleanser, a furniture polish, an automobile polish, and a cleanser for other solid surfaces. Various embodiments of the invention such as lotions, soaps, creams, aerosols, gels, and medicinal lotions, may be suitable for application to consumer items like facial and toilet tissue and to personal care products like diapers, training pants, feminine hygiene products and adult care products.

The materials desirably have both inorganic and organic fragments in their framework. The definitions of “inorganic” and “organic” here refer to traditionally accepted meanings for these two terms defined by common chemistry text books, namely: “organic” is related to carbon-based and associated with “life” while “inorganic’ covers all other atoms and their chemistry. Materials having carbon-metal bonds are often called “organometallics” and are also considered suitable for use in the invention. For the sake of simplicity and clarity, any compound or material that has both “inorganic” and “organic” fragments is described herein as a “hybrid” material.

The suitable molecular-level multi-functional ingredients for the current invention can be selected from the group consisting of a polyhederal oligomeric silsequioxane (POSS), zeolite, cyclomacroether, porphyrin, foldamer, dendrimers, cyclodextrin and mixtures thereof. Various substituents or moieties may be attached to these nanostructured materials for reaching multifunctional benefits. These reactive moieties include alkyls, alcohols, alkoxysilanes, amines, chlorosilanes, epoxides, esters, halides, methacrylates, molecular silicas, nitriles, norbornenes, olefins, phosphines, silanes, silanols, styrenic polymers, polyolefins, and mixtures thereof. The reactive moiety may be bonded directly to the nanostructured compound or may be bonded through an organic, siloxane or organosiloxane group.

The nanostructured compound may desirably be a POSS compound or derivative having a cage composed of silicon and oxygen, and having a molecular formula of:

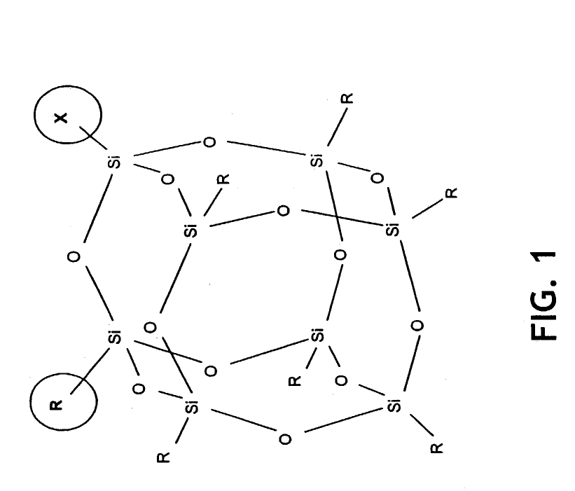

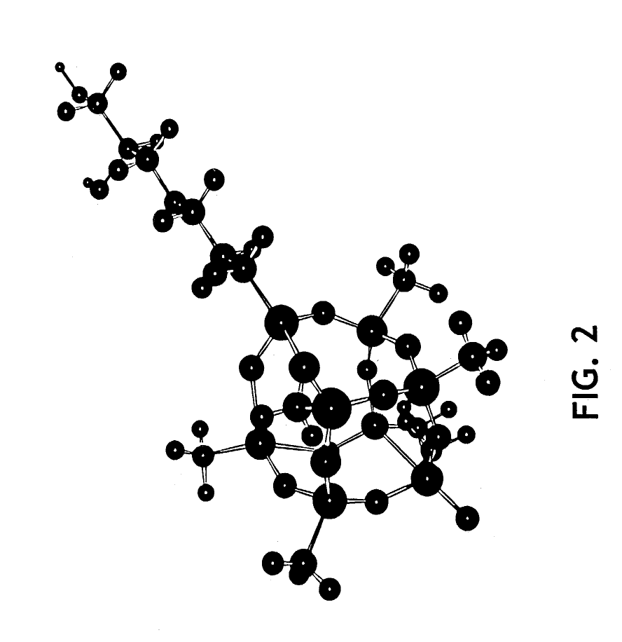

SiₓO_{3/2x}Rₓ

where x ranges from 4 to 60, and R is a moiety defined above which may be bonded directly to the silicon atoms forming the cage. Desirably, x is 4, 8, 10, 12 and/or 14. A description of possible cages is discussed in U.S. Pat. No. 5,942,638 and various academic articles such as Silsequioxane-Siloxane Copolymers from Polyhedral Silsesquioxanes in the journal Macromolecules, 1993, 26, p 2141-2142. Each of the cages can be further modified by attaching reactive moieties to the cage atoms. FIG. 1 is a representative sketch of a POSS hybrid having molecular-level functional ingredients. The R and X groups can be modified to be either hydrophobic or hydrophilic. FIG. 2 is a three dimensional drawing of a multi-functional POSS compound with hydrophilic groups on the cage and hydrophilic OH group on the end of the arm. The cage area is suitable for small molecule such as oxygen to pass and also refract UV light. POSS hybrid chemical compounds are commercially available from Hybrid Plastics™ of Fountain Valley, Calif. (www.hybridplastics.com).

Examples of POSS compounds include but not limited to following:

1-[3-(allylbisphenol A)propyldimethylsiloxy]-3,5,7,9,11,13,-15heptacyclopentylpentacyclo-[9.5.1.13,9.15,15.17,13]octasiloxane;

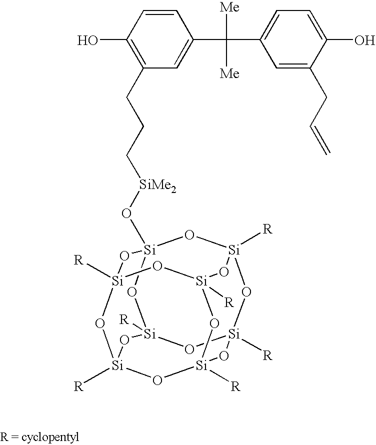

1-[3-(allylbiphenol)propyidimethylsiloxy] 3,5,7,9,11,13,15heptacyclopentylpentacyclo-[9.5.1.13,9.15,15.17,13]octasiloxane;

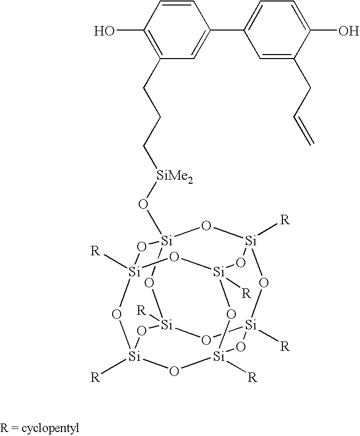

1-[3-(1,3-propanediol-2-ethyl-2-methyloxy)propyidimethylsiloxy]-3,5,7,9,11,13,15-heptacyclopentylpentacyclo-[9.5.1.13,9.15,15.17,13]octasiloxane;

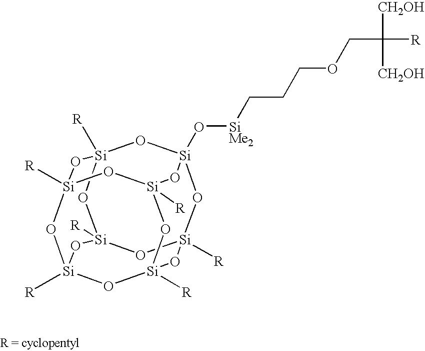

1-[(2-methyl,2-hydroxy)butyldimethylsiloxy]-3,5,7,9,11,13,15-heptacyclopentylpentacyclo-[9.5.1.13,9.15,15.17,13]octasiloxane

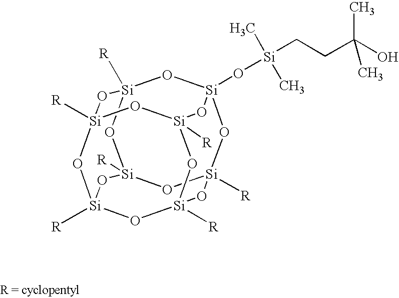

1-[3-(ethoxydimethylsilyl)propyl]3,5,7,9,11,13,15-heptacyclopentylpentacyclo[9.5.1.13,9.15,15,17,13]octasiloxane

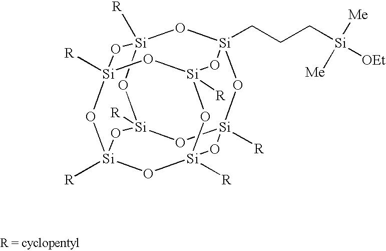

1-[2-(diethoxymethylsilyl)propyl]5,7,9,11,13,15-heptacyclopentylpentacyclo[9.5.1.13,9.15,15,17,13]octasiloxane:

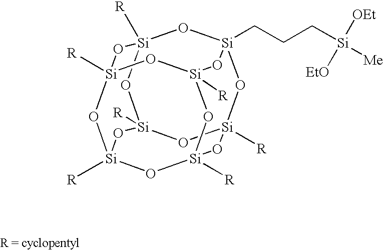

1-[3-(triethoxysilyl)propyl]3,5,7,9,11,13,15-heptacyclopentylpentacyclo[9. 5.1.13,9.15,15,17,13]octasiloxane;

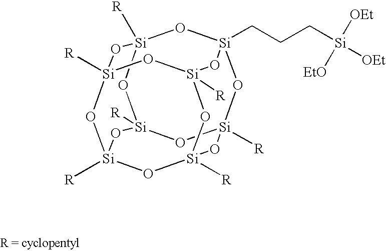

1-[2-(ethoxydimethylsilyl)ethyl]3,5,7,9,11,13,15-heptacyclopentylpentacyclo[9.5.1.13,9.15,15,17,13]octasiloxane

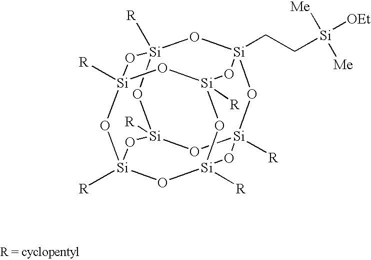

1-[2-(diethoxymethylsilyl)propyl]3,5,7,9,11,13,15-heptacyclopentylpentacyclo[9.5.1.13,9.15,15,17,13]octasiloxane;

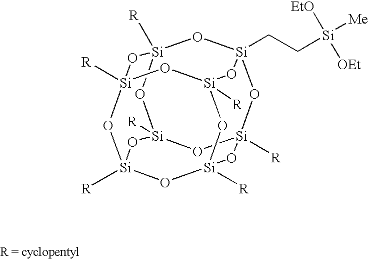

1-[2-(triethoxysilyl)propyl]3,5,7,9,11,13,15-heptacyclopentylpentacyclo[9. 5.1.13,9.15,15,17,13]octasiloxane;

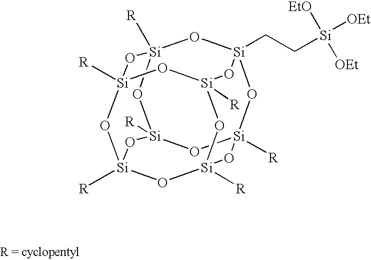

POSS-BisPhenol A-urethanes

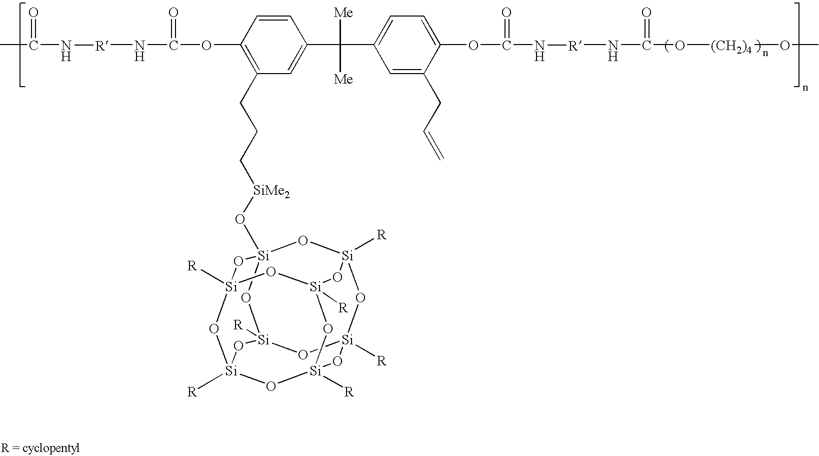

POSS-DiMethylol-urethanes;

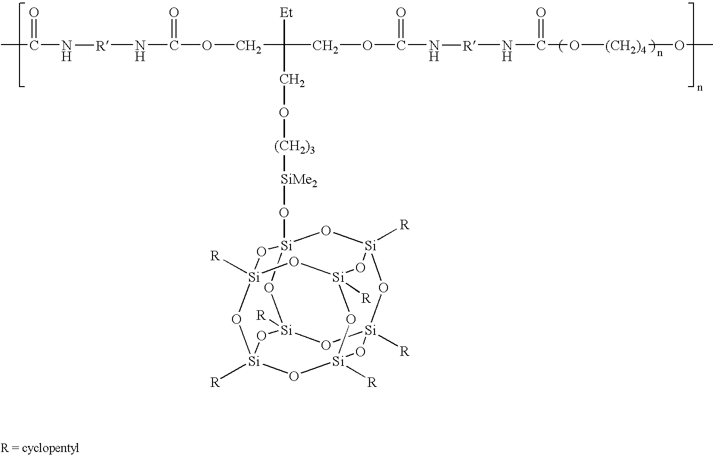

1-chloro-3,5,7,9,11,13,15-heptacyclopentylpentacyclo[9.5.1.13,9.15,15. 17,13]octasiloxane;

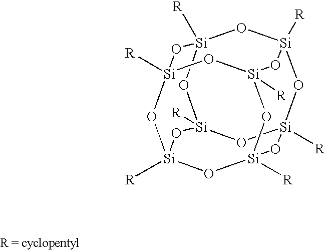

1-[2-(chlorodimethylsilyl)ethyl]-3,5,7,9,11,13,15heptacyclopentylpentacyclo[9.5.1.13,9.15,15.17,13]octasiloxane;

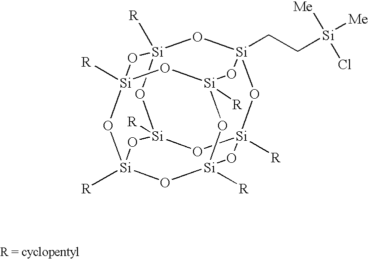

1-[2-(dichloromethylsilyl)ethyl]-3,5,7,9,11,13,15heptacyclopentylpentacyclo[9.5.1.13,9.15,15.17,13]octasiloxane;

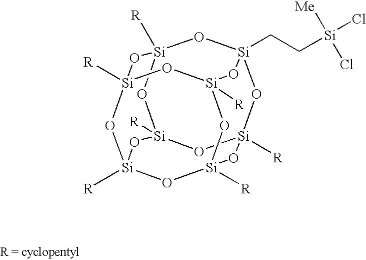

1-[2-(trichlorosilyl)ethyl]-3,5,7,9,11,13,1 5-heptacyclopentylpentacyclo[9. 5.1.13,9.15,15.17,13]octasiloxane;

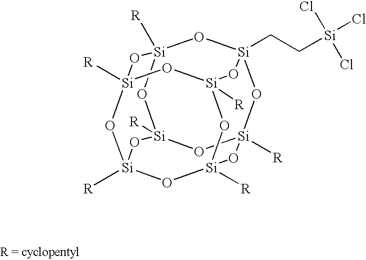

1-[3-(chlorodimethylsilyl)propyl]-3,5,7,9,11,13,15-heptacyclopentylpentacyclo[9.5.1.13,9.15,15.17,13]-octasiloxane;

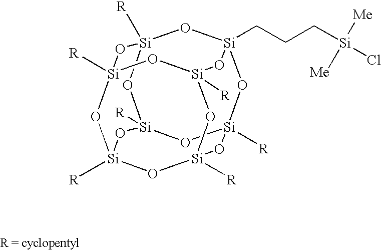

1-[3-(dichloromethylsilyl)propyl]-3,5,7,9,11,13,15-heptacyclopentylpentacyclo[9.5.1.13,9.15,15.17,13]-octasiloxane;

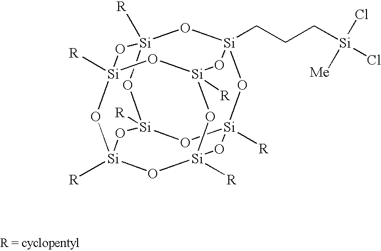

1-[3-(trichlorosilyl)propyl]-3,5,7,9,11,13,15-heptacyclopentylpentacyclo[9.5.1.13,9.15,15.17,13]octasiloxane;

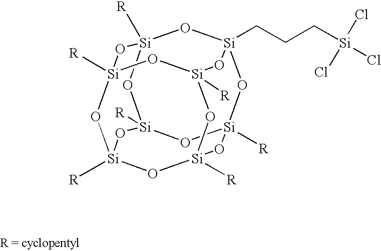

1,3,5,7,9,11,13,15-[2-(chlorodimethylsilyl)ethyl]pentacyclo[9.5.1.1 3,9.15,15.17,13]octasiloxane;

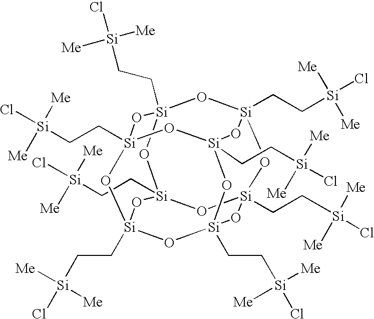

1,3,5,7,9,11,13,15-[2-(chlorodimethylsilyl)ethyl]pentacyclo[9.5.1.1 3,9.15,15.17,13]octasiloxane;

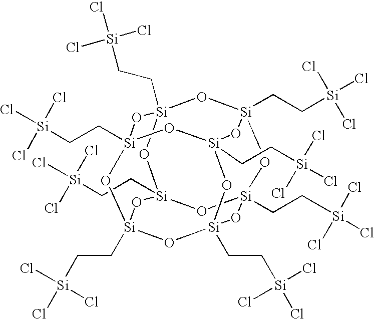

1,3,5,7,9,111,13,15-[2-(dichlorodimethylsilyl)ethyl]pentacyclo[9.5.1.13,9.15,15.17,13]octasiloxane;

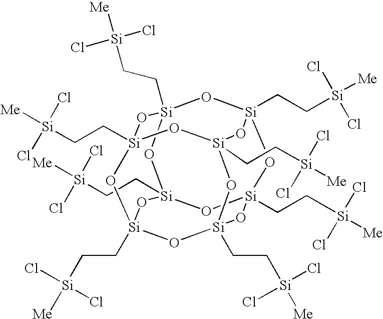

1-[(2-epoxy)propyl]-3,5,7,9,11,13,15-heptacyclopentylpentacyclo[9.5.1.13,9.15,15.17,13]octasiloxane;

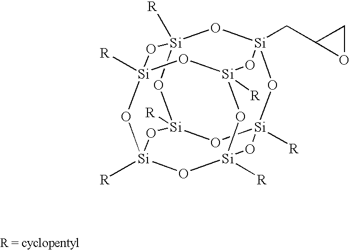

1-[2-(cyclohexyl-3-epoxy)ethyl]-3,5,7,9,11,13,15-heptacyclopentylpentacyclo[9.5.1.13,9.15,15.17,13]-octasiloxane;

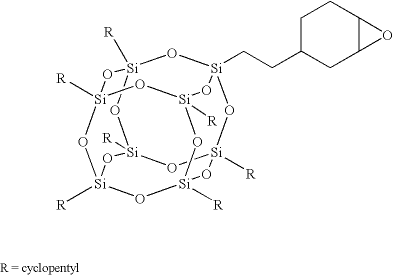

POSS-diepoxide resins;

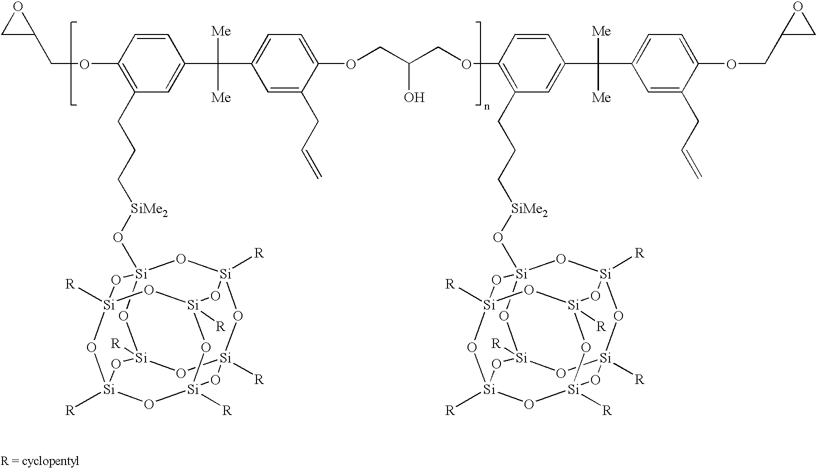

1,3,5,7,9-octavinyl-11,13,15-epoxyethylpentacyclo[9.5.1.1.3,9.1. 15,15.1.17,13]octasiloxane;

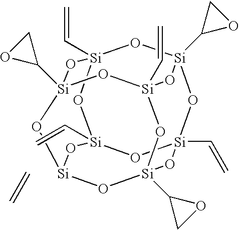

endo-3,7,14-tris[1-(3-dimethylsiloxy)propyloxy-2,3--epoxypropyl]-1,3,5,7,9,11,14,-heptacyclopentyltricyclo[7.3.3.1,5,11]-heptasiloxane;

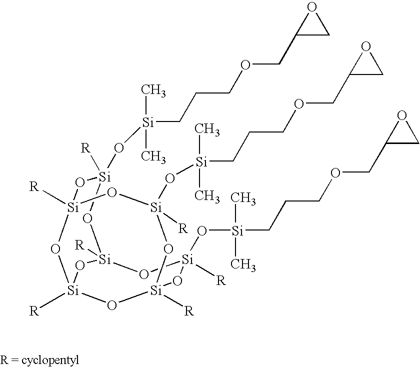

1-(methylpropionato)-3,5,7,9,11,13,15-heptacyclopentylpentacyclo[9.5.1.1. sup.3,9.15,15.17,13]octasiloxane;

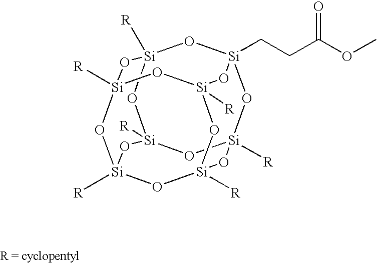

1-(ethylundecanoato)-3,5,7,9,11,13,15-heptacyclopentylpentacyclo[9.5.1.1. sup.3,9.15,15.17,13]octasiloxane;

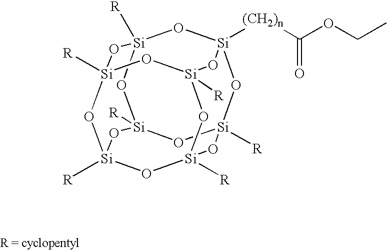

1-[(3-chloro)propyl]-3,5,7,9,11,13,15-heptacyclopentylpentacyclo[9.5.1. 13,9.15,15.17,13]octasiloxane;

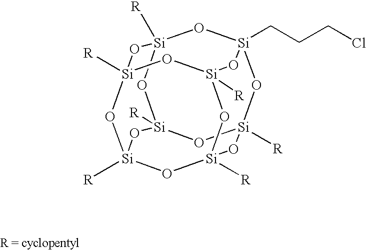

1-[4-chlorophenyl]-3,5,7,9,11,13,15-heptacyclopentylpentacyclo[9.5.1.13,9. 15,15.17,1 3]octasiloxane

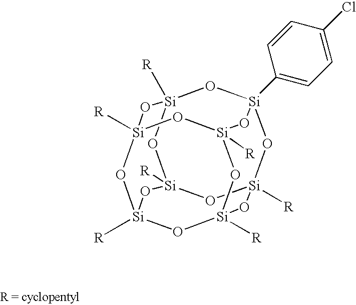

1-[chlorobenzyl]-3,5,7,9,11,13,15-heptacyclopentylpentacyclo[9.5.1.13,9. 15,15.17,13]octasiloxane;

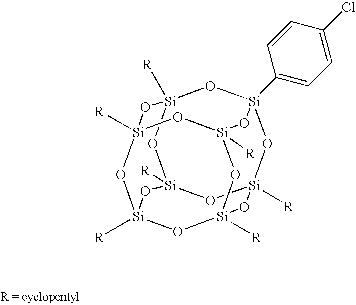

1-[2-(chlorobenzyl)ethyl]-3,5,7,9,11,13,15-heptacyclopentylpentacyclof[9.5. 1.13,9.15,15.17,13]octasiloxane;

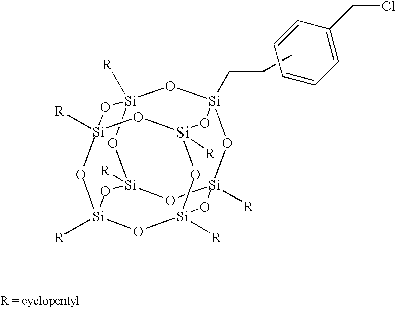

1-[3-(methacryl)propyl]-3,5,7,9,11,13,15-heptacyclopentacyclo[9.5.1 13,9.15,15.17,13]octasiloxane;

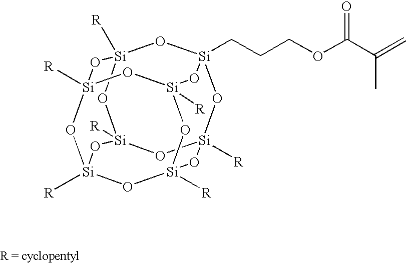

1-[3-(methacryl)propyldimethylsiloxy]-3,5,7,9,11,13,15-heptacyclopentylpentacyclo[9.5.1.13,9.15,15.17,13]-octasiloxane;

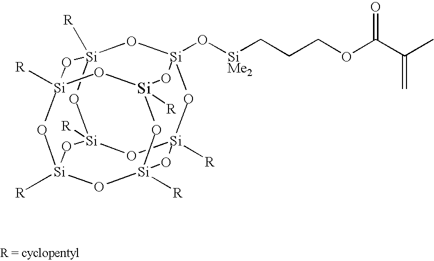

1-(3,3,3-trifluoropropyidimethylsiloxy)-1,3,5,9,11,13,15-heptacyclopentyl-7-[3-(methacryl)propyl]-7-methyltetracyclo[9.5.1.15,11.19,15]octasiloxane;

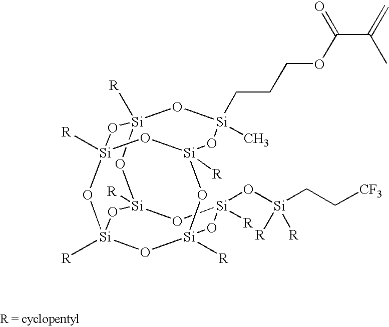

1-(tridecafluoro-1,1,2,2-tetrahydrooctyldimethylsiloxy)-1,3,5,9,11,13,15-heptacyclopentyl-7-[3-(methacryl)propyl]-7-methyltetracyclo[9.5.1.15,11.19,15]octasiloxane;

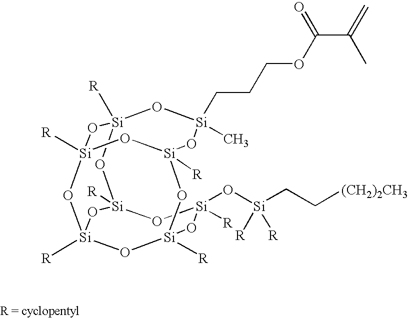

1-(trimethylsiloxy)-1,3,5,9,11,13,15-heptacyclopentyl-7-[3-(methacryl)propyl]-7-methyltetracyclo[9.5.1.15,11.19,15]octasiloxane;

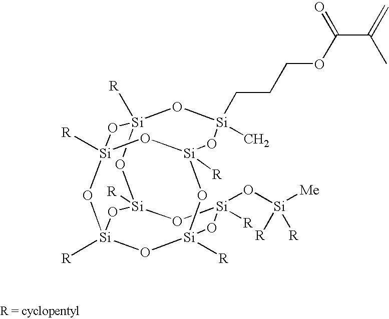

1,3,5,7,9-pentavinyl-11,13,15-[1-hydroxy-2-(methacryl)ethyl]pentacyclo[9.5.1.13,9.15,15.17,13]octasiloxane;

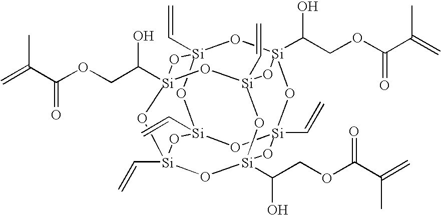

1,3,5,7,9,11-hexacyclohexyltetracyclo[5.5.1.13,11.15,9]hexasiloxane;

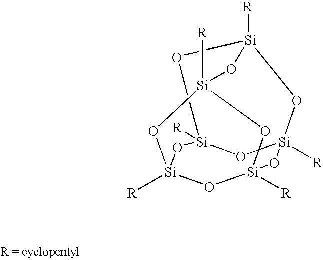

1,3,5,7,9,11,13,15-octacyclohexylpentacyclo[9.5.1.13,9.15,15. 17,13]octasiloxane

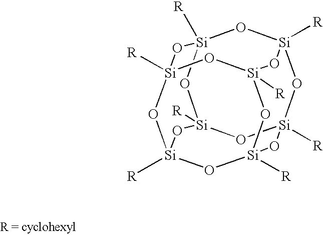

1,3,5,7,9,11,13,15-octacyclopentylpentacyclo[9.5.1.13,9.15,15.17,13]octasiloxane;

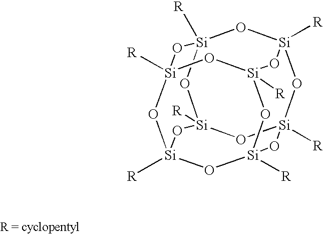

1,3,5,7,9,11,13,15-octaphenylpentayclo[9.5.1.13,9.15,15.17,13, ]octasiloxane;

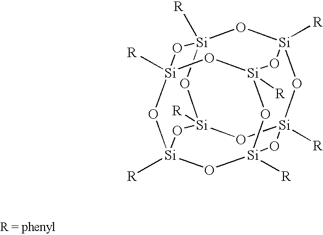

1,3,5,7,9,11,13,15-octamethylpentayclo[9.5.1.13,9.15,15.17,13, ]octasiloxane;

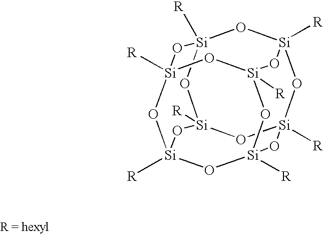

1,3,5,7,9,11,13,15-octakis(dimethylsilyloxy)pentacyclo[9.5.1.13,9.1. sup.5,15.17,13]octasiloxane;

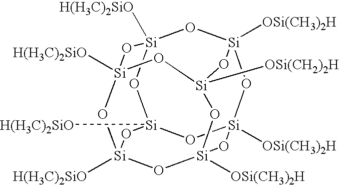

POSS-modified Nylon 6;

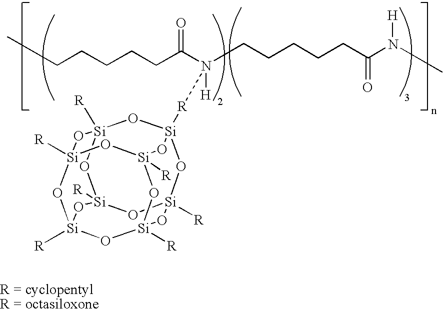

1-[(3-cyano)propyl]-3,5,7,9,11,13,15-heptacyclopentylpentacyclo[9.5.1.1. sup.3,9.15,15.17,13]octasiloxane;

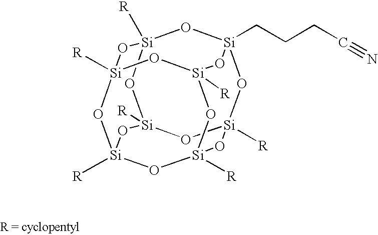

1-[2-(Norbornen-2-yl)ethyl]-3,5,7,9,11,13,15-heptacyclopentylpentacyclo[9. 5.1.13,9.15,15.17,13]octasiloxane;

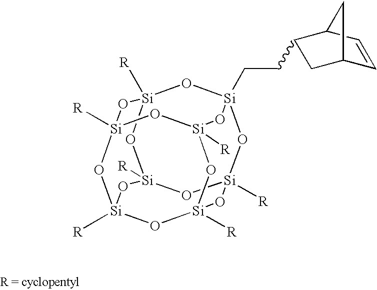

1-[2-(Norbornen-2-yl)ethyldimethylsiloxy]-3,5,7,9,11,13,15heptacyclopentylpentacyclo[9.5.1.13,9.15,15.17,13]-octasiloxane;

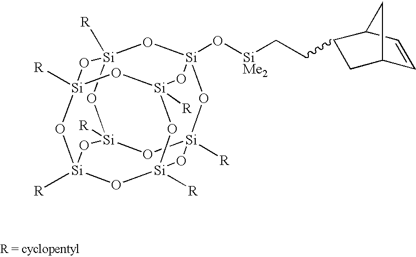

Poly(ethylnorbomenylPOSS-co-norbornene);

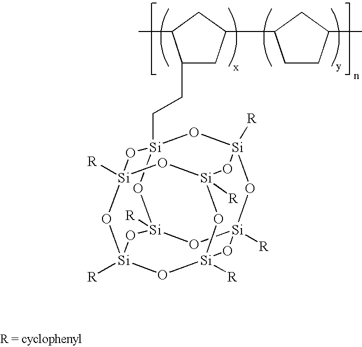

1,1,3,3-(Norbornenyldimethylsiloxy)-1,3,-dicyclohexyldisiloxane

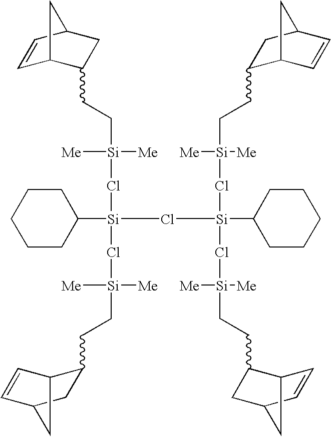

1-[3-(allylbisphenol A)propyldimethylsiloxy]-3,5,7,9,11,13,15heptacyclopentylpentacyclo-[9.5.1.13,9.15,15.17,13]octasiloxane;

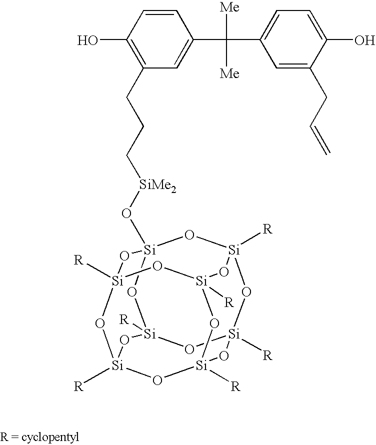

1-[3-(allylbiphenol)propyldimethylsiloxy]-3,5,7,9,-11,13,15heptacyclopentylpentacyclo-[9.5.1.13,9.15,15.17,13]octasiloxane;

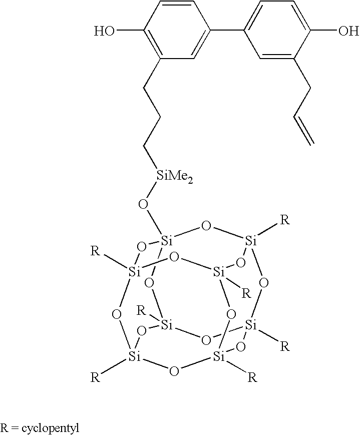

1,3,5,7,9,11,13,15-octavinylpentacyclo[9.5.1.13,9.15,15.17,1 3]octasiloxane;

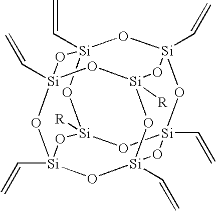

1-vinyl-3,5,7,9,11,13,15-heptacyclopentylpentacyclo[9.5.1.13,9.15,15.17,13]octasiloxane;

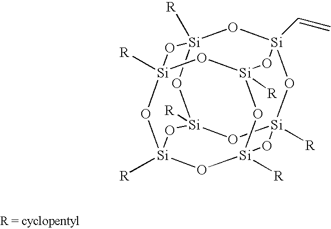

1-allyl-3,5,7,9,11,13,15-heptacyclopentylpentacyclo[9.5.1.13,9.15,15.17,13]octasiloxane

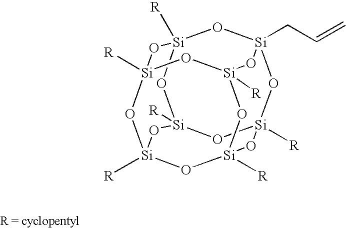

1-[2-(cyclohexen-3-yl)ethyl]-3,5,7,9,11,13,15-heptacyclopentylpentacyclo[9.5.1.13,9.15,15.17,13]octasiloxane;

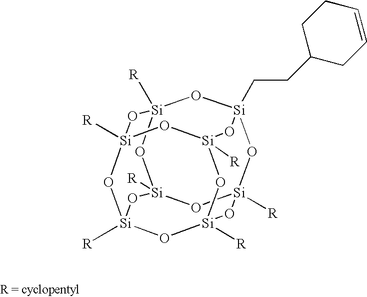

Poly(dimethyl-co-methylvinyl-co-methylethylsiloxyPOSS)siloxane;

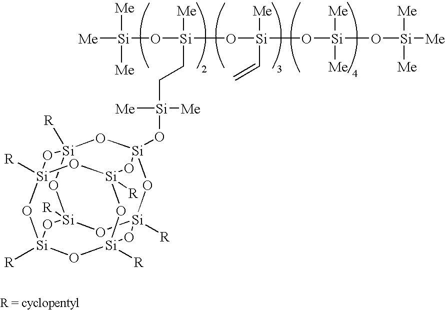

POSS-diepoxide resins;

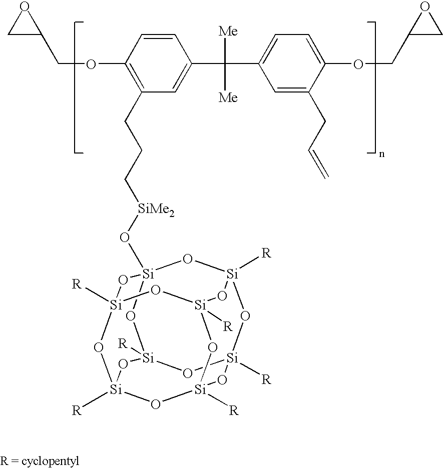

POSS-BisPhenol A-urethanes;

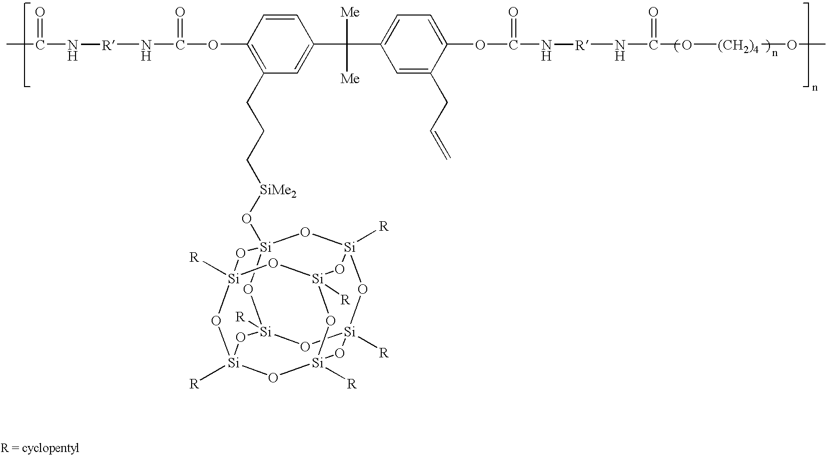

1-[2(diphenylphosphino)ethyl]3,5,7,9,11,13,15-heptacyclopentylpentacyclo[9.5.1.13,9.15,15.17,13]octasiloxane;

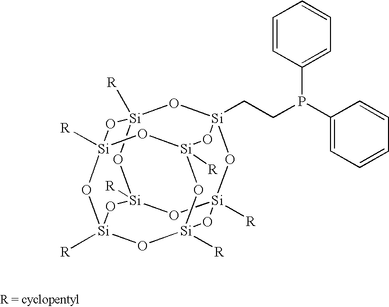

1-[2(diphenylphosphino)propyl]3,5,7,9,11,13,15-heptacyclopentylpentacyclo[9.5.1.13,9.15,15.17,13]octasiloxane;

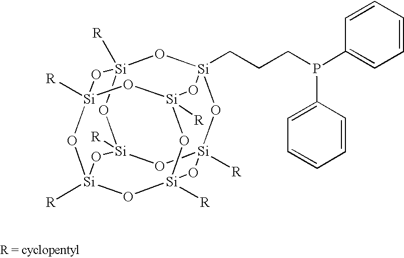

1-hydrido-3,5,7,9,11,13,15-heptacyclopentylpentacyclo[9.5.1.13,9.15,15. 17,13]octasiloxane;

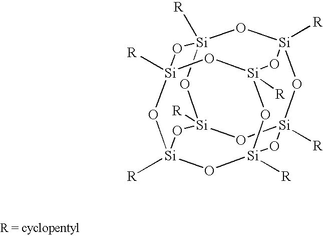

1-[hydridodimethylsiloxy]-3,5,7,9,11,13,15-heptacyclopentylpentacyclo[9.5. 1.13,9.15,15.17,13]octasiloxane

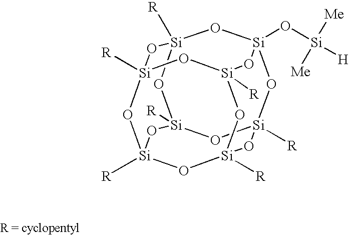

endo-3,7,14-tri(dimethylsilylhydrido)-1,3,5,7,9,11,14-heptacycopentyltricyclo[7.3.3.15,15,11]heptasiloxane;

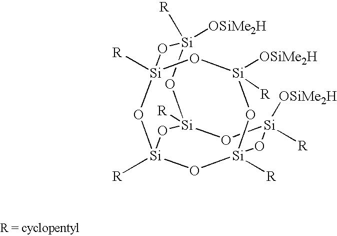

1,1,3,3-(hydridodimethylsiloxy)-1,3-dicyclohexyldisiloxane

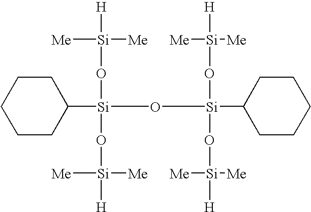

Poly(dimethyl-co-methylhydrido-co-methylpropylPOSS)siloxaneendo-3,7,14-trihydroxy-1,3,5,7,9,11,14-heptacyclopentyltricyclo[7.3.3. 15,11]heptasiloxane;

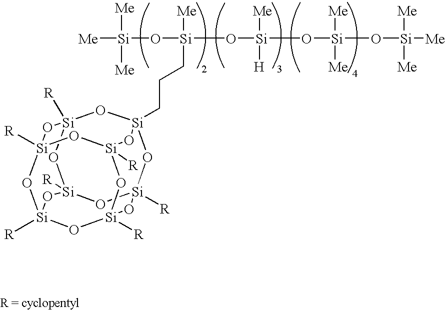

endo-3,7,14-trihydroxy-1,3,5,7,9,11,14-heptacyclohexyltricyclo[7.3.3.15,11]heptasiloxane;

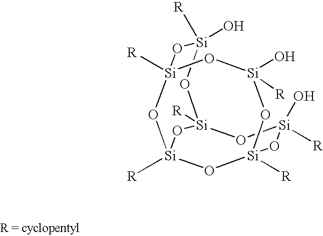

1-hydroxy-3,5,7,9,11,13,15-heptacyclopentylpentacyclo[9.5.1.13,9.15,15. 17,13]octasiloxane; 1,1,3,3-(tetrahydroxy)-1,3-dicyclohexyldisiloxane;

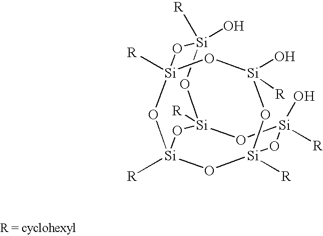

1,3,5,7-(tetrahydroxy)-1,3,5,7-(tetraphenyl)cyclotetrasiloxane;

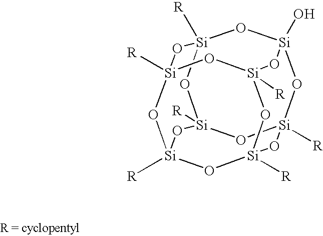

endo-7,14-dihydroxy-3-(3,3,3-trifluoropropyldimethylsiloxy)-1,3,5,9,11,13,15-heptacyclopentyltricyclo[7.3.3. 15,11]octasiloxane;

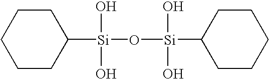

,3,5,7-(tetrahydroxy)-1,3,5,7, -(tetraphenyl)cyclotetrasiloxane

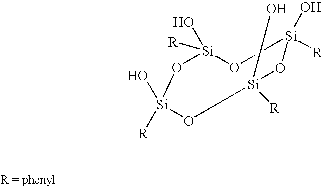

endo-7,14,-dihydroxy-3-(3,3,3-trifluoropropyldimethylsiloxy)-1,3,5,9,11,13,15-heptacyclopentyltricyclo[7.3.3.1^{5,11}]octasiloxane

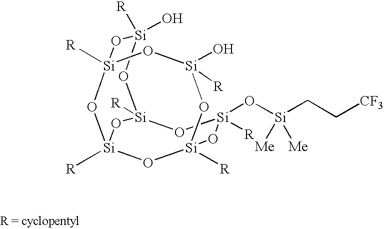

1-[2-(styryl)ethyldimethylsiloxy]-3,5,7,9,11,13,15-heptacyclopentylpentacyclo[9.5.1.13,9.15,15.17,13]-octasiloxane;

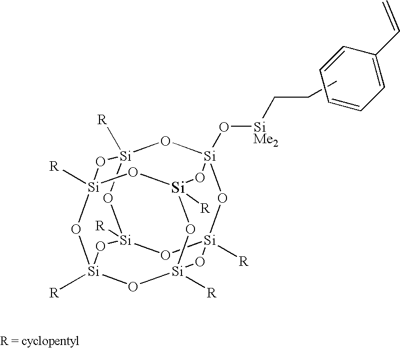

1-[(4-vinyl)phenyl]-3,5,7,9,11,13,15-heptacyclopentylpentacyclo[9.5.1.13,9.15,15.17,13]octasiloxane

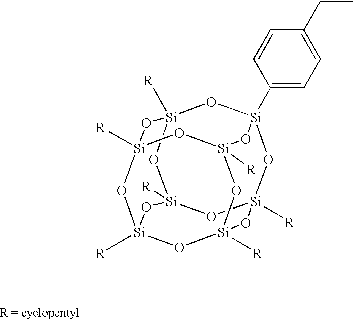

1-[2-(styryl)ethyl]-3,5,7,9,11,13,15-heptacyclopentylpentacyclo[9.5.1.13,9.15,15.17,13]-octasiloxane;

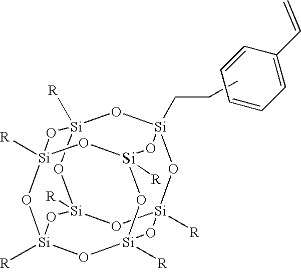

R=cyclopentyl, TMP DiolCyclopentyl-POSS

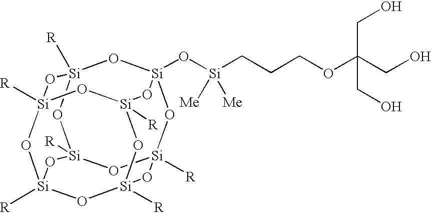

R=i-butyl, Trans-CyclohexaneDiollsobutyl-POSS

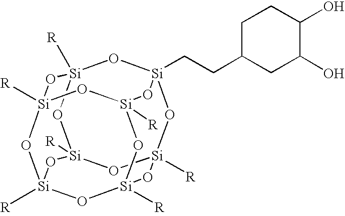

R=i-butyl, 1,2-PropaneDiollsobutyl-POSS

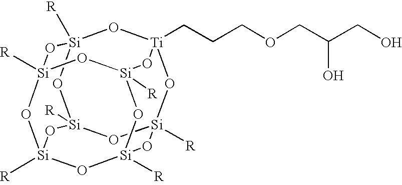

R=i-butyl, Aminopropyllsobutyl-POSS

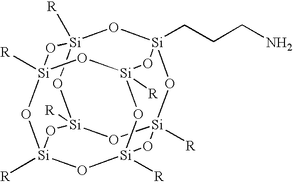

R=i-octyl, Aminopropyllsobutyl-POSS

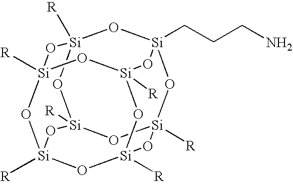

R=i-butyl, Aminoethylaminopropyllsobutyl-POSS

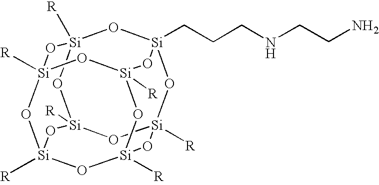

R=Cyclopentyl, IsocyanatopropyldimethylsilylCyclopentyl-POSS

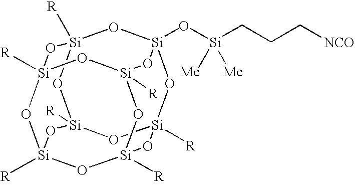

R=i-butyl, MethacryllsobutylTitanium-POSS

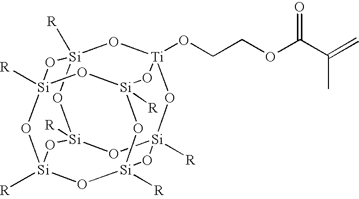

OctaAmmonium-POSS

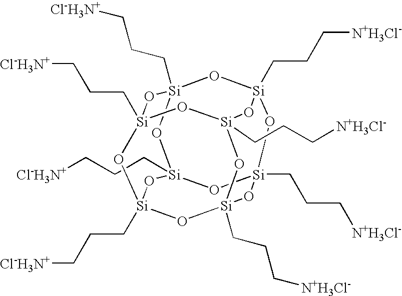

OctaAmmonium-POSS

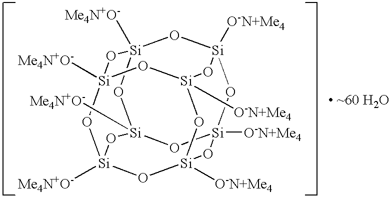

Poly(styrylPOSS-co-styrene)

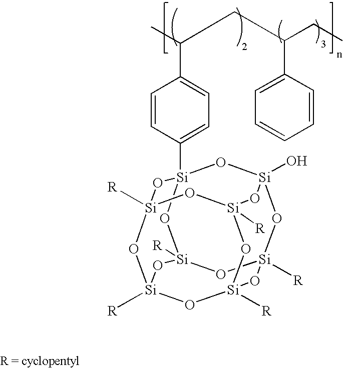

poly(vinylsilsesquioxane)

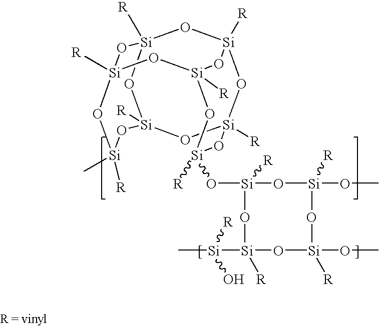

and structures having 10 and 12 silicon atoms in the cage.

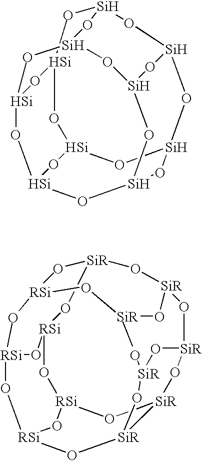

In another desired embodiment, the molecular-level hybrid ingredients are covalently bonded to a larger molecule, for example, as a pendant group on an organic or organosiloxane polymer. In addition or in the alternative, the hybrid compound is a repeat unit in an oligomer or polymer. An example of oligomeric binding of the POSS is as follows:

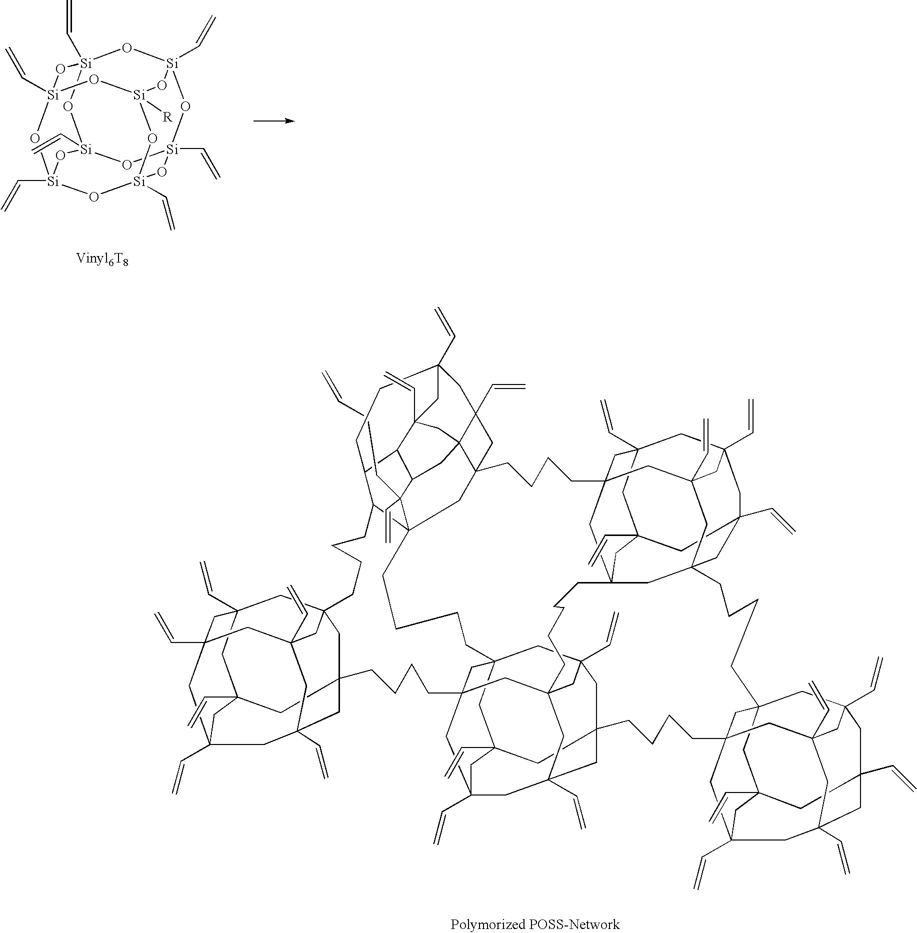

It's desired to include molecular-level nanostructured POSS compounds into a composition to provide a breathable protection layer on the skin.

Conventional methods of producing a breathable layer depend on very complex physical and chemical theories, notably membrane laws by Fick, and solution-diffusion of gases in polymers. Zeolites used as molecular sieves require additional work, such as pressure changes, or temperature sieving manipulations to effect gas permeation. The main limiting factor is that the size of the opening through which the skin nutrients, such as oxygen, must pass, cannot be controlled or structured to eliminate hole size from being a variable in the “permeation coefficient”. It is thus advantageous to form a breathable surface (e.g. skin) protection layer with high uniformity and regularity.

POSS compounds are capable of enhancing small molecule separation for the passage of oxygen through the skin, though the breathability of a POSS containing protection layer is not limited to only oxygen. Other small molecules such as nitrogen, carbon dioxide, and water are also permeable through such layers. Such properties of POSS compositions allow the maintenance of a “pseudo” natural environment for the skin even with a protection layer.

It's believed that POSS compounds create a rigid “backbone” of inorganic material within the organic molecular structure of monomers and polymers, separating the organic components and actually creating a small hole in the organic material. The hole sizes are on the order of Angstroms and can be varied over an appreciable range, depending upon the combination of the specific POSS compound and the monomer or polymer selected. If more selectivity or “fine tuning” of the hole sizes is required, the POSS compounds can be used with organic materials that possess considerable elasticity. If such materials are stretched, the holes in the material become elongated in the direction of stretching, and become smaller in the direction normal to the stretching force. The elasticity of such POSS compositions is important because surface temperature or conditions may vary according to the environmental changes.

As noted above, various moieties may be attached to the nanostructured materials. The composition for breathability may also be a blend of at least two different polymers or may be a random or block copolymer of at least two different polymeric segments. The composition may have at least one polymeric segment selected from the group consisting of mineral oil, petrolatum, silicon oils, polyvinyl, polycarbonate, polyurethane, poly(diorgano)siloxane, polysulfone, polyamide, poly(epoxide), polyepichlorohydrin, polyether, polyester, polyketone, and polyalkylene. Wherein the organo-group of the poly(diorgano)siloxane is selected from the group consisting of methyl, ethyl, n-propyl, isopropyl, n-butyl, iso-butyl, tert-butyl, pentyl, hexyl, cyclohexyl and phenyl; wherein the polyvinyl is selected from the group consisting of polyvinyl alcohol, poly(vinyl alcohol-co-ethylene), polyvinyl chloride, polyvinyl bromide, poly(vinyl acetate), poly(alkyl)acrylate, poly(alkyl)methacrylate, poly(acrylic acid) or salt thereof, polyacrylonitrile, polystyrene, poly(vinyl sulfonic acid) or salt thereof, and poly(vinyl methyl ketone); wherein the polyether is selected from the group consisting of poly(ethylene glycol), poly(propylene glycol), poly(ethylene terephthalate), poly(ethylene succinate), polyacetal, and polytetrahydrofuran; and wherein the polyalkylene is selected from the group consisting of polyethylene, polypropylene and polybutadiene. In another aspect, In the preparation of the breathable protection composition, the POSS nanostructured compound may be bonded through a reactive group to the composition.

In another aspect, it's desired to provide a molecular-level topical protection layer with formulations that contain POSS compounds and that are breathable. POSS compounds not only can be modified to have a reactive coupling group for attachment on the surface but also can use their cages/caged polymer networks to provide multiple benefits such as the delivery of a bioactive agent. In POSS compounds, the reactive X groups can be modified and formulated to be able to readily react and chemically bond to the surfaces (e.g. skin). The chemically bonded POSS layer on the surface then behaves as a delivery system while providing other protection functions such as, for example, UV radiation. In this manner, it is possible for POSS to provide for the long-term adhesion and slow-release of various bioactive agents on the surface of human skin. Agents activated for reaction with the skin surface and that bind the bioactive agent to the skin surface enhances safety for many bioactive agents. The bioactive agents may be used to act as sunscreens, antimicrobials, deodorants, skin protectants, skin moisturizers, and for enhanced repellency to microorganisms, chemicals, insect bites, water, plant irritants such as poison ivy/oak, and other skin irritants. Other effects such as artificial skin coloring and administration of topical drugs, among others may also be possible.

Antimicrobial agents include phenol, creosols, hydrozybenzoates, resorcinol, 4-hexylresorcinol, hexachlorophene, triclosan, salicylanilide, tetracycline, quinolones, bacitracin, gramicidin, polymyxin B, streptomycin B, neomycin A, erythromycin, gentamicin, chlorhexidine, pyrithione, miconazole, thimersal, triclocarban, cloflucarban and various penicillins.

In another aspect of the invention, the use of POSS compounds brings another benefit: the “hollow sphere” effect, to the formulations in which they are contained. The hollow sphere effect refers to the ability reflect and refract light. Latex hollow sphere nanoparticles can effectively enhance the sun protection factor (SPF) numbers by as high as 80 percent as noted in U.S. Pat. No. 5,663,213, to Rohm &Haas (Philadelphia, Pa.). The '213 patent discloses sunscreen formulations that use SunSphere® hollow particles to enhance SPF numbers. Although it's advantageous to use latex particles to produce the hollow sphere effect, it's even more advantageous to have a molecular-level hollow sphere effect. The caged nanostructures of POSS compounds allows them to be ideal molecular-level hollow spheres.

The hollow sphere effect of the POSS cages reflects some of the light away from the skin of a wearer, for example, or refracts it towards UV absorbers in the balance of a sun protection formulation. UV absorbers suitable for use with POSS include oxybenzone, dioxybenzone, sulisobenzone, methyl anthranilate, para-aminobenzoic acid, amyl paradimethyaminobenzoate, ethyl 4-bis(hydroxypropyl)para-aminobenzoate, diethanolamine para-methyoxycinnamate, 2-ethoxyethyl para-methoxycinnamate, ethylhexyl para methoxycinnamate, octyl paramethoxycinnamate, isoamyl para-methoxycinnamate, 2-ethylhexyl 2 cyano-3, 3-diphenyl-acrylate, 2-ethylhexyl salicylate, homomenthyl salicylate, glyceryl aminobenzoate, triethanolamine salicylate, digalloyl trioleate, lawsone with dihydroxyacetone, 2-phenylbenzimidazole-5-sulfonic acid, benzylidine camphor, avobenzone, titanium dioxide and zinc oxide.

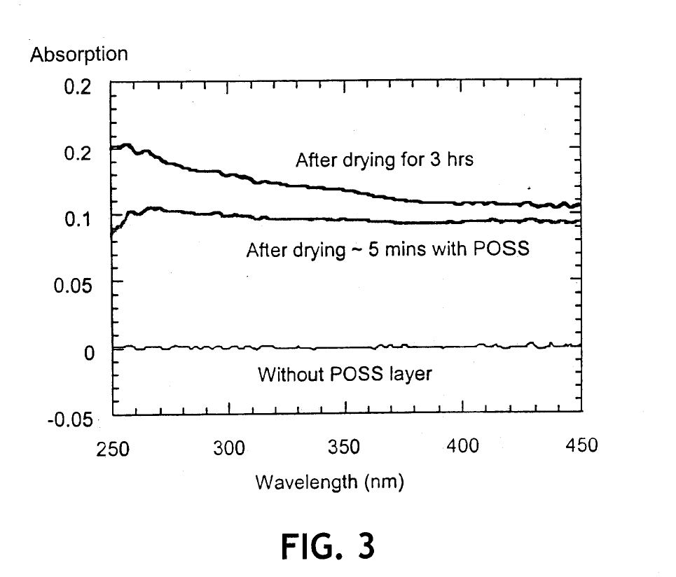

In order to demonstrate the possible hollow sphere effect, a solution of a POSS compound was dissolved into a hydrocarbon pentane solution and coated onto quartz glass slide as discussed in Example 2 below. After evaporation of the pentane solvent, a UV range absorption spectrum was taken and the result is shown in FIG. 3. In further demonstrating such an effect, a sunscreen was prepared and the SPF number was measured and compared to the same formulation without POSS. The results clearly demonstrated the UV wavelength light scattering properties of this type of compound. Light scattering properties are desired for many cosmetics.

POSS compounds can be formulated either through oil phase or water phase mixing, though the predominate phase of the composition is usually water. It should be noted that the current invention is suitable but not limited to two phase systems such as water-in-oil or oil-in-water systems. Multiple emulsion systems as well as molecular-level emulsion systems are also desired for producing such formulations.

Desirably, water soluble POSS compounds are formulated into the water phase of the compositions and hydrocarbon soluble POSS compounds are formulated into the oil phase of compositions. In cases where no suitable solvents are available, they can be formulated as a suspension. For some embodiments, heat may be necessary to accelerate the dissolving process.

Suitable oils for use in the compositions of the present invention include all of the conventional water-insoluble liquid or semi-solid mineral (including mixtures of petroleum derived hydrocarbons containing 10 or more carbon atoms), vegetable, synthetic and animal oils, including fatty acid esters, lanolin, etc. The particular oils selected for a composition will depend upon its desired function and/or its ability to dissolve additives such as fragrances, waxes, medicaments, etc.

Suitable waxes include semi-solid and solid vegetable, mineral, animal and synthetic waxes, present in amounts which are soluble in the oils present to form liquid oil/wax mixtures.

POSS containing formulations containing both water and hydrocarbons desirably have between 10 and 90 weight percent water, from a positive amount to 50 weight percent POSS with the balance hydrocarbon, more desirably between about 20 and 60 weight percent water, between about 2 and 30 weight percent POSS, and hydrocarbon, more desirably between about 35 and 45 weight percent water, between about 2 and 10 weight percent POSS, and hydrocarbon.

POSS compounds that have alkyl hydrocarbon groups have great solubilities in volatile organic solvents such as pentanes, hexanes, heptanes, and mixtures thereof. Accordingly, the current invention adds other benefits to the POSS containing formulations by combining volatile hydrocarbons with POSS compounds to provide foaming capabilities. The desired volatile organic foam-producing liquids are those having a vapor pressure from about 967-1484.2 mmHg (4 to 14 psig) at temperatures between about 32-38° C. (90 to 100° F.), most desirably hydrocarbons having 5 or 6 carbon atoms such as isopentane, pentane and hexane. The volatile organic liquid may be present in an amount between about 1 and 20 weight percent, desirably between 7 and 12 weight percent.

The volatility of low boiling point organic solvents helps to create the foams upon vaporization and can be used as an indicator for when and if cleaning is complete. The foaming function is a positive innovation for consumers who want to know if cleaning is complete, and also for parents/teachers/caregivers who want to educate/train children to follow cleaning procedures in the home, school, public areas, etc. The combination of POSS compounds and volatile organic solvents not only provides foaming but also may provide molecule-level protection benefits such as breathability and UV protection, discussed above.

POSS compounds and organic foaming agents may be combined with other foaming technologies such as dispensing as an aerosol to add special effects for the foaming process. Air trapped in the formulation, for example, may more effectively bring volatile organic solvents to the surface for enhanced foaming. Such a combination may also create a sensation of cooling on the skin upon applying the foaming composition. The use of low boiling point solvents, furthermore, assures the rapid formation of a skin protection layer. As a functional ingredient carrier, low boiling point solvents will quickly precipitate functional ingredients onto a surface, because they are the first to vaporize in the formulation. This provides an easy and practical way to design formulations that can selectively form a protection layer on the skin, depending on the solubility of functional ingredients in these solvents.

The POSS compounds can in some cases be considered as a surfactant if their cages are hydrophobic and their arms are hydrophilic. In such cases they enable the presence of large amounts of oil or oil/wax in water, in fact, this may allow the incorporation of from about 5 to about 60 weight percent of the oil or oil/wax, desirably from 20 to about 35 weight percent of the oil or oil/wax, in water as an emulsion. While various POSS compounds may function as a surfactant, additional surfactants may be necessary depending on the POSS compound chosen and the type and amount of hydrocarbon and water to be included in the composition, as well as other factors. If present, a non-POSS surfactant may be used in an amount up to about 15 weight percent, more desirably about 10 weight percent.

Surfactants contain hydrophilic and hydrophobic segments that increase the solubility of the hydrocarbons in water. In some cases this may result in excessive foaming, sometimes requiring foam suppressing agents or anti-foams. The most desired mild surfactants that usually do not need foam suppressing agents include anionic materials such as sodium methyl cocoyl taurate (sodium salts of N-methyl taurine-coconut oil amides, referred to herein as SMCT); disodium cocamido monoisopropanol amide sulfosuccinate (disodium salts of sulfo-2-cocamide-1-methyl ethyl esters of butanedioic acid, referred to herein as DCMS); sodium lauryl sulfoacetate (sodium salt of sulfo-1-dodecyl ester of acetic acid, referred to herein as SLSA); dioctyl sodium sulfosuccinate (sodium salt of the diester of 2-ethylhexyl alcohol and sulfosuccinic acid, referred to herein as DSS), and amphoteric materials such as cocamidopropyl hydroxy sultaine, referred to herein as CHS, and combinations of such surfactants with each other or with small amounts of other foam-boosting surfactants.

While some surfactants by their nature do not need the addition of anti-foams, other surfactants are not suitable for commercial utilization without them. In cases where more than 10 to 15 weight percent hydrocarbons are used for the foaming compositions, the addition of foam-suppressing compounds or detergents may increase the solubility of the volatile hydrocarbon component. The inclusion of foam-suppressing compounds, desirably in amounts between about 3 and 7 weight percent where necessary, in combination with highly foaming surfactants, allows the use of a greater variety of surfactants which, alone, are not useful for the preparation of suitable commercial products. Combinations of this type can be obtained by adding alcohols, alkoxy alcohols, and so forth to surfactants like sodium lauryl sulfate, referred to herein as SLS, ammonium lauryl ether sulfate, referred to herein as ALS, and many other surfactants generally of the anionic type. A similar foam-controlling effect can be obtained by blending high foaming surfactants, generally of the anionic type, with low foaming surfactants. The desired low foaming surfactants used for this foam controlling effect are generally of the non-ionic and the amphoteric type. Typical of these are nonoxinol-9 (nonionic) and cocampho-carboxyl glycinate (amphoteric), referred to herein as CCG.

The desired foam suppressing agents are alcohols, alkoxy alcohols, low foaming surfactants and any other suitable compounds that help prevent the compositions of the invention from self-dispensing at temperatures well in excess of room temperature.

In still another embodiment of the invention, the foaming control may be combined with color. Colors can be added in solvents in either the oil or water phase. Colored foams are more appealing, especially for children in training for cleaning habits. Desirably, color dyes for the current invention are environmentally safe and do no harm to applied surfaces (e.g. skin, etc). Color producing compounds suitable for use in this invention include those US Food and Drug Administration (FDA) approved color additives such as D&C Green No.8, FD&C Blue No.1, carotene and so on. The full list of color additives used for cosmetics can be found in the FDA website (http://www.cfsan.gov/˜dms/opa-col2.html). If used, dyes may be present in a minor amount of the composition, desirably less than about 5 weight percent.

More desirably, dyes used in the practice of the current invention can change color upon vaporization of the volatile organic solvents. The dye may be colorless in hydrocarbons, for example, and turn to other colors (e.g. blue, red, etc) upon removal of the solvents. Dyes showing different colors with different solvent compositions are suitable for this application, and examples can be found in U.S. Pat. No. 4,824,827. The vaporization of the volatile organic solvents may be controlled to occur after a predetermined, finite time through the careful choice of solvents. In this manner, the color-change time may be controlled to coincide with the typical amount of time that should be spent in a particular activity. Hand cleaning soaps, for example, may be formulated such that the volatile organics are vaporized and the color changes after one or two minutes, indicating to the user that the proper hand cleaning interval has been reached.

Other ingredients generally are present in minor amounts; less than 10 weight percent and usually less than about 2 weight percent. Such additives desirably are soluble in or miscible with either the oils or oil/wax mixtures or in the water. Suitable additives include perfumes or fragrances, emollients, humectants, medicaments, colorants, etc.

In one exemplary formulation, the inventive composition may contain at least about 5 weight percent of one or more oils, alone or in combination with one or more waxes soluble in or compatible with the oil(s), and POSS compounds. Optionally, at least about 5 weight percent of at least one suitable surface active agent or surfactant, at least about 5 weight percent of at least one volatile organic foam-producing liquid, and/or one or more foam-suppressing compounds in an amount up to 15 weight percent may be included. Other optional ingredients like colorants may also be included. The balance is water.

The following examples are illustrative of several compositions within the scope of the present invention and should not be considered limiting. The compositions are produced by adding the oils or oil/wax mixtures to a suitable container, adding the other materials thereto and homogenizing at a suitable elevated temperature until the ingredients are thoroughly dispersed in the oil or oil/wax phase, with water being added while homogenizing. The mixture is cooled to room temperature or lower and the optional volatile organic foam-producing liquid, cooled below its boiling point, may be added while the composition is stirred with a mixer. Generally any additives are incorporated with the oils or oil/wax mixtures in the first step.

### EXAMPLE 1

This example describes the procedure to formulate POSS compounds with hydrocarbons as a solvent.

A pentane solution was prepared by dissolving 1 gram of POSS-OH (i-butyl, 1,2-PropaneDiollsobutyl-POSS )in 3 grams of pentane. Any hydrocarbon soluble POSS compound would also be suitable, but the amount of solvent may be different. The resulting solution may be used directly as one phase to be formulated into lotions, creams, soaps and so forth.

It should be noted here that other solvents (halides, esters, acids, ethers, and etc) may also be used, depending on specific applications.

### EXAMPLE 2

This example describes the procedure to measure UV absorption by a layer of POSS compounds on a hard surface, in this case a quartz slide. SPF numbers were recorded by a. Labsphere UV Transmittance Analyzer, model no. UV1000, from the Labsphere Incorporation of North Sutton, N.H. 03260.

A clean quartz slide was flushed (only one side) with the solution of a POSS containing hydrocarbon solvent of Example 1 and then air dried. The resulting milk-colored slide surface was placed under an SPF analyzer and its UV absorption spectrum was recorded. An SPF increase of 20˜34% was observed, as shown in FIG. 3. It was found that prolonged drying further enhanced the absorption.

### EXAMPLE 3

This example describes the procedure to formulate POSS compounds with a solvent mixture of hydrocarbon and mineral or silicon oils. Since POSS compounds are not readily soluble in mineral or other similar oils at room temperature and even elevated temperatures (up to 80° C.), an alternative approach was used to formulate them into an oil phase. In this procedure, a POSS compound was first dissolved into a small amount of hydrocarbon solvent and then mixed with desired amount of mineral oil and other oil phase ingredients.

In a typical experiment, 1 gram of hydrocarbon soluble POSS compound was first dissolved into 3 grams of pentane (or hexane, heptanes, etc) and then mixed with 10 grams of mineral oil. After thorough mixing, this oil phase may be mixed with other oil phase ingredients.

The ratios of POSS and mineral oil can be controlled in a range of 0.025:99.9, depending on the desired application.

### EXAMPLE 4

This example describes the procedure to formulate POSS compounds as a suspension in oil phase. In case no suitable solvent is available to dissolve POSS compounds, they can be formulated by suspension. This can be done either by mixing with other solid powders such as titanium dioxide or mixed directly.

A POSS containing oil phase was prepared by using following compositions and procedure: A mixture of mineral oil (12 gram), petrolatum (3 gram), isopropyl myristate (3 gram), and octyl dimethyl PABA (4 gram) were mixed first at 70° C. and then mixed with a POSS-OH powder. The resulting oil phase can be used to form lotions and other desired formulations.

### EXAMPLE 5

This example describes the procedure to formulate water-soluble POSS compounds. A base solution in the water phase was prepared by mixing the following: water (64 grams), sodium chloride (2 grams), and glycerin (3 grams), then 1 gram of octa-POSS ammonium salt was dissolved into the above mixture by vigorous stirring. The resulting POSS containing water phase can be used to formulate water-in-oil or oil-in-water based lotions and like.

### EXAMPLE 6

This example describes the procedure to formulate a foaming sunscreen with POSS compounds. Phase A as described below without TINO-40 and avobenzone, was heated to 80° C. The TINO-40/C12-15 alkyl benzoate and avobenzone were then slowly added. The resulting oil phase was next added to phase B at 80° C., stirred for 20 minutes and then allowed to cool to room temperature. Finally the phase C was added to the mixture and rigorously mixed until the formulation became homogeneous.

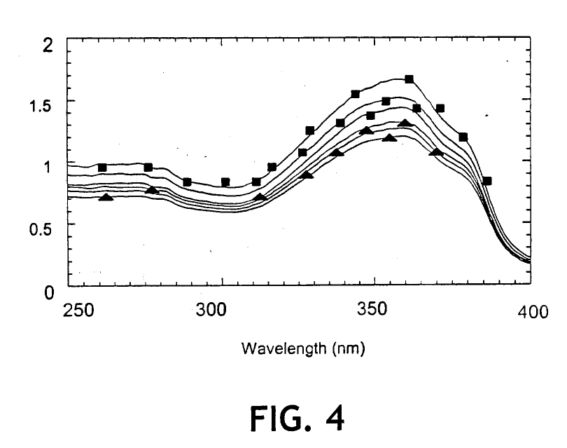

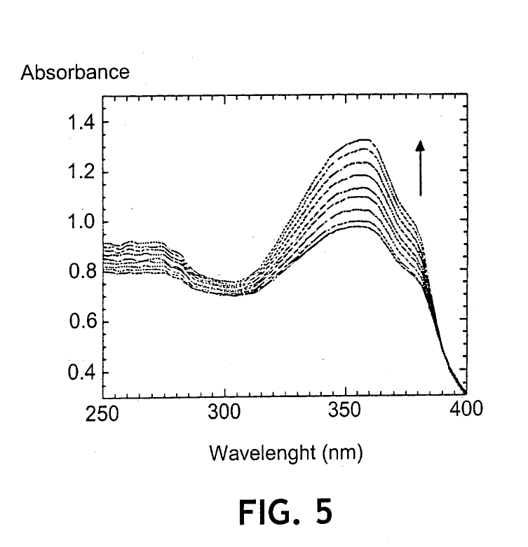

FIG. 4 shows the testing of the material of Example 6 and a composition differing only in that no POSS was added to it. The POSS containing formulations had higher UV absorbance than those without POSS. FIG. 5 shows testing of the formulation of Example 6 at equal time intervals over a half hour period. As the formulation dried, the absorbance increased.

| A (Oil Phase): | |
|---|---|
| Cetearyl alcohol | 4 g |
| Finosolv | 2 g |
| Mineral Oil | 4 g |
| Stearic acid | 2 g |
| TINO-40 (Altair, Nevada)/C12-15 alkyl benzoate (40% dispersion) | 5 g |
| Avobenzone | 3 g |

| B (Water Phase): | | |
|---|---|---|
| Deionized (DI) water | 76 | g |
| Carbomer | 0.1 | g |
| Triethylene amine | 0.4 | g |
| Methyl paraben | 0.1 | g |
| Disodium EDTA | 0.05 | g |
| C(POSS containing phase) | | |
| Neopentane | 10 | g |
| POSS-OH | 3 | g |

Examples 7 to 31 are a List of Possible Formulations that may Contain POSS

### EXAMPLE 7

| Ingredients | Wt % |
|---|---|
| Mineral Oil | 35.0 |
| DCMS | 15.0 |
| POSS | 5.0 |
| Water | 35.0 |
| Pentane | 10.0 |

### EXAMPLE 8

| Ingredients | Wt % |
|---|---|
| Mineral Oil | 35.0 |
| DCMS | 13.0 |
| Sodium Lauryl Sulfate | 2.0 |
| POSS | 5.0 |
| Fragrance | 0.5 |
| Water | 34.5 |
| Isopentane | 10.0 |

### EXAMPLE 9

| Ingredients | Wt % |
|---|---|
| Mineral Oil | 30.0 |
| SMCT | 15.0 |
| POSS | 5.0 |
| Water | 40.0 |
| Pentane | 10.0 |

### EXAMPLE 10

| Ingredient | Wt % |
|---|---|
| Mineral Oil | 30.0 |
| POSS | 5.0 |
| DCMS | 20.0 |
| Water | 35.0 |
| Pentane | 10.0 |

### EXAMPLE 11

| Ingredients | Wt % |
|---|---|
| Mineral Oil | 30.0 |
| POSS | 5.0 |
| DCMS | 5.0 |
| Water | 50.0 |
| Pentane | 10.0 |

### EXAMPLE 12

| Ingredients | Wt % |
|---|---|
| Petroleum Jelly | 15.0 |
| POSS | 5.0 |
| Mineral Oil | 10.0 |
| Isopropyl Palmitate | 10.0 |
| DCMS | 12.0 |
| SLS | 3.0 |
| Water | 39.5 |
| Fragrance | 0.5 |
| Isopentane | 10.0 |

### EXAMPLE 13

| Ingredients | Wt % |
|---|---|
| Mineral Oil | 30.0 |
| POSS | 5.0 |
| SLSA | 5.0 |
| Water | 50.0 |
| Isopentane | 10.0 |

### EXAMPLE 14 (SHAVING PREPARATION)

| Ingredients | Wt % |
|---|---|
| Mineral Oil | 15 |
| POSS | 5 |
| Isopropyl Palmitate | 8 |
| Glycerine | 10 |
| DCMS | 13 |
| SLS | 2 |
| Palmitic Acid | 2 |
| Water | 35 |
| Isopentane | 10 |
| NaOH to adjust to pH 8.5 | |

### EXAMPLE 15 (ANTIDUNDRUFF CLEANSER)

| Ingredient | Wt % |
|---|---|
| Mineral Oil | 35.0 |
| POSS | 5.0 |
| DCMS | 15.0 |
| Omadine MDS (MgSO.sub.4 adduct | 1.0 |
| Water | 34.0 |
| Isopentane | 10.0 |

### EXAMPLE 16 (PSORIATIC SKIN CLEANSER)

| Ingredient | Wt % |
|---|---|
| Petroleum Jelly | 10.0 |
| Mineral Oil | 20.0 |
| POSS | 5.0 |
| DCMS | 15.0 |
| Coal Tar Aqueous Soln. | 5.0 |
| Water | 35.0 |
| Isopentane | 10.0 |

### EXAMPLE 17 (AUTO CLEANER/POLISH)

| Ingredient | Wt % |
|---|---|
| Carnauba Wax | 17.5 |
| Mineral Oil | 17.5 |
| POSS | 5.0 |
| DCMS | 9.0 |
| SLS | 1.0 |
| Water | 40.0 |
| Isopentane | 10.0 |

### EXAMPLE 18 (FABRIC CLEANER)

| Ingredient | Wt % |
|---|---|
| Deodorized Kerosen | 30.0 |
| POSS | 5.0 |
| DCMS | 15.0 |
| Water | 40.0 |
| Pentane | 5.0 |

### EXAMPLE 19 (COSMETIC CLEANSER)

| Ingredient | Wt % |
|---|---|
| Lanolin | 10.0 |
| POSS | 5.0 |
| Acetulan (acetylated lanolin) | 25.0 |
| DCMS | 13.0 |
| SLS | 2.0 |
| Water | 40.0 |
| Isopentane | 10.0 |

### EXAMPLE 20

| Ingredient | Wt % |
|---|---|
| DCMS | 15.0 |
| Mineral Oil | 45.0 |
| POSS | 5.0 |
| Water | 15.0 |
| Isopentane | 10.0 |

### EXAMPLE 21

| Ingredients | Wt % |
|---|---|
| DCMS | 5.0 |
| Mineral Oil | 35.0 |
| POSS | 5.0 |
| Water | 45.0 |
| Isopentane | 10.0 |

### EXAMPLE 22

| Ingredients | Wt % |
|---|---|
| SCT | 15.0 |
| Mineral Oil | 10.0 |
| POSS | 5.0 |
| Water | 60.0 |
| Isopentane | 10.0 |

### EXAMPLE 23

| Ingredients | Wt % |
|---|---|
| DSS | 15.0 |
| Mineral Oil | 35.0 |
| POSS | 5.0 |
| Water | 35.0 |
| Isopentane | 10.0 |

### EXAMPLE 24

| Ingredients | Wt % |
|---|---|
| DCMS | 12.0 |
| SLS | 3.0 |
| Mineral Oil | 20.0 |
| POSS | 5.0 |
| Petroleum Jelly | 12.5 |
| Polyethyleneglycol-8 | 2.5 |
| Dilaurate | |
| Water | 34.5 |
| Fragrance | 0.5 |
| Hexane | 10.0 |

### EXAMPLE 25 (DEPILATORY LOTION)

| Ingredients | Wt % |
|---|---|
| DCMS | 15.0 |
| Mineral Oil | 35.0 |
| POSS | 5.0 |
| Water | 34.0 |
| Calcium Thioglycolat | 6.0 |
| Isopentane | 10.0 |

### EXAMPLE 26

| Ingredients | Wt % |
|---|---|
| SLS | 15.0 |
| Mineral Oil | 30.0 |
| POSS | 5.0 |
| Water | 35.0 |
| Ethyleneglycol | 5.0 |
| n-Butyl ether | |
| Pentane | 10.0 |

### EXAMPLE 27

| Ingredients | Wt % |
|---|---|
| SLS | 15.0 |
| Mineral Oil | 30.0 |
| POSS | 5.0 |
| Water | 33.0 |
| Ethanol | 7.0 |
| Pentane | 10.0 |

### EXAMPLE 28

| Ingredients | Wt % |
|---|---|
| SLS | 7.5 |
| Cocampho-carboxyl | 7.5 |
| glycinate (CCG) | |
| Mineral Oil | 30.0 |
| POSS | 5.0 |
| Water | 40.0 |
| Pentane | 10.0 |

### EXAMPLE 29

| Ingredients | Wt % |
|---|---|
| SLS | 7.5 |
| Nonoxinol-9 | 7.5 |
| Mineral Oil | 30.0 |
| POSS | 5.0 |
| Water | 40.0 |
| Pentane | 10.0 |

### EXAMPLE 30

| Ingredients | Wt % |
|---|---|
| CHS | 15.0 |
| Mineral Oil | 35.0 |
| POSS | 5.0 |
| Water | 35.0 |
| Pentane | 10.0 |

### EXAMPLE 31 (SURGICAL SCRUB)

| Ingredients | Wt % |
|---|---|
| CHS | 15.0 |
| Chlorhexidine gluconate | 4.0 |
| Capramide DEA | 2.0 |
| Polyquaternium-7 | 0.2 |
| Mineral Oil | 18.0 |
| Petroleum Jelly | 12.0 |
| POSS | 5.0 |
| Fragrance | 0.3 |
| Water | 34.5 |
| Pentane | 10.0 |

### EXAMPLE 32 (ACNE CLEANSER)

| Ingredients | Wt % |
|---|---|
| DCMS | 12.0 |
| ALS | 3.0 |
| Polyquaternium-7 | 0.2 |
| Mineral Oil | 25.0 |
| POSS | 5.0 |
| Benzoyl Peroxide | 5.0 |
| Glycerine | 5.0 |
| Water | 34.5 |
| Fragrance | 0.3 |
| Pentane | 10.0 |

As will be appreciated by those skilled in the art, changes and variations to the invention are considered to be within the ability of those skilled in the art. Examples of such changes are contained in the patents identified above, each of which is incorporated herein by reference in its entirety to the extent it is consistent with this specification. Such changes and variations are intended by the inventors to be within the scope of the invention.

What is claimed is:

1. A composition for surface cleaning and protection comprising a molecular-level multi-functional ingredient having organic and inorganic portions and selected from the group consisting of polyhederal oligomeric silsequioxanes (POSS) compounds, zeolites, cyclomacroethers, porphyrins, foldamers, dendrimers, cyclodextrins and derivatives and mixtures thereof.

2. The composition of claim 1 further comprising a moiety attached to said molecular-level multi-functional ingredient wherein said moiety is selected from the group consisting of alkyls, alcohols, alkoxysilanes, amines, chlorosilanes, epoxides, esters, halides, methacrylates, molecular silicas, nitrites, norbornenes, olefins, phosphines, silanes, silanols, styrenic polymers, polyolefins and mixtures thereof.

3. The composition of claim 1 wherein said reactive moiety is bonded directly to said molecular-level multi-functional ingredient.

4. The composition of claim 1 wherein said reactive moiety is bonded to said molecular-level multi-functional ingredient through an organic, siloxane or organosiloxane group.

5. The composition of claim 1 wherein said molecular-level multi-functional ingredient is a POSS compound having a cage composed of silicon and oxygen, and having a molecular formula of Siₓ O_{3/2x} Rₓ where x ranges from 4 to 60, and R is a moiety selected from the group consisting of alkyls, alcohols, alkoxysilanes, amines, chlorosilanes, epoxides, esters, halides, methacrylates, molecular silicas, nitriles, norbornenes, olefins, phosphines, silanes, silanols, styrenic polymers, polyolefins, and mixtures thereof, which is bonded directly to said silicon atoms forming said cage.

6. The composition of claim 5 wherein x is a number selected from the group consisting of 4, 8, 10, 12 and 14.

7. The composition of claim 5 which is present in a product selected from the group consisting of lotions, creams, aerosols, gels, soaps, medicinal lotions, fabric cleansers, furniture polishes, automobile polishes, and a cleanser for surfaces.

8. The composition of claim 5 further comprising a volatile organic foam-producing liquid in an amount between about 1 and 20 weight percent.

9. The composition of claim 5 further comprising a volatile organic foam-producing liquid in an amount between about 7 and 12 weight percent.

10. The composition of claim 8 further comprising a color dye.

11. The composition of claim 10 wherein said color changes after a predetermined time interval.

12. The composition of claim 5 which is present in a product selected from the group consisting of lotions, creams, aerosols, gels, soaps, and medicinal lotions which is applied to an item selected from the group consisting of facial tissue, toilet tissue, diapers, training pants, feminine hygiene products and adult care products.

13. A composition comprising water in an amount of between about 10 and 90 weight percent and a POSS compound in an amount of between a positive amount and 50 weight percent, and hydrocarbon.

14. The composition of claim 12 comprising between about 20 and 60 weight percent water, between about 2 and 10 weight percent POSS, and hydrocarbon.

15. The composition of claim 12 further comprising a surfactant in an amount of between about a positive amount and 10 weight percent.

16. The composition of claim 14 further comprising a foam suppressing compound in an amount of between about 1 and 15 weight percent.

17. The composition of claim 15 further comprising a color dye.

18. The composition of claim 16 wherein said color changes after a predetermined time interval.

19. A composition to deliver active agents to skin comprising a POSS compound having a cage and further comprising a bioactive agent selected from the group consisting of sunscreens, antimicrobials, deodorants, skin protectants, skin moisturizers, artificial skin coloring, topical drugs and repellents to microorganisms, chemicals, insect bites, water, plant irritants and other skin irritants.

20. The composition of claim 19 wherein said bioactive agent is a repellent to microorganisms, chemicals, insect bites, water, and plant irritants

21. The composition of claim 19 wherein said bioactive agent is an antimicrobial selected from the group consisting of phenol, creosols, hydrozybenzoates, resorcinol, 4-hexylresorcinol, hexachlorophene, triclosan, salicylanilide, tetracycline, quinolones, bacitracin, gramicidin, polymyxin B, streptomycin B, neomycin A, erythromycin, gentamicin, chlorhexidine, pyrithione, miconazole, thimersal, triclocarban, cloflucarban and various penicillins.

22. The composition of claim 21 wherein said antimicrobial is triclosan.

23. The composition of claim 21 wherein said antimicrobial is neomycin A.

24. The composition of claim 21 wherein said antimicrobial is bacitracin.

25. The composition of claim 19 wherein said bioactive agent is a sunscreen which includes a UV absorbing agent selected from the group consisting of oxybenzone, dioxybenzone, sulisobenzone, methyl anthranilate, para-aminobenzoic acid, amyl paradimethyaminobenzoate, ethyl 4-bis(hydroxypropyl)para-aminobenzoate, diethanolamine para-methyoxycinnamate, 2-ethoxyethyl para-methoxycinnamate, ethylhexyl para methoxycinnamate, octyl paramethoxycinnamate, isoamyl para-methoxycinnamate, 2-ethylhexyl 2 cyano-3,3-diphenyl-acrylate, 2-ethylhexyl salicylate, homomenthyl salicylate, glyceryl aminobenzoate, triethanolamine salicylate, digalloyl trioleate, lawsone with dihydroxyacetone, 2-phenylbenzimidazole-5-sulfonic acid, benzylidine camphor, avobenzone, titanium dioxide, zinc oxide and mixtures thereof.

26. The composition of claim 25 wherein said sunscreen includes titanium dioxide.

27. The composition of claim 25 wherein said sunscreen includes zinc oxide.

28. A composition for breathable skin protection comprising a POSS compound having at least one polymeric segment selected from the group consisting of mineral oil, petrolatum, silicon oils, polyvinyl, polycarbonate, polyurethane, poly(diorgano)siloxane, polysulfone, polyamide, poly(epoxide), polyepichlorohydrin, polyether, polyester, polyketone, and polyalkylene, wherein the organo-group of the poly(diorgano)siloxane is selected from the group consisting of methyl, ethyl, n-propyl, isopropyl, n-butyl, iso-butyl, tert-butyl, pentyl, hexyl, cyclohexyl and phenyl, the polyvinyl is selected from the group consisting of polyvinyl alcohol, poly(vinyl alcohol-co-ethylene), polyvinyl chloride, polyvinyl bromide, poly(vinyl acetate), poly(alkyl)acrylate, poly(alkyl)methacrylate, poly(acrylic acid) or salt thereof, polyacrylonitrile, polystyrene, poly(vinyl sulfonic acid) or salt thereof, and poly(vinyl methyl ketone), the polyether is selected from the group consisting of poly(ethylene glycol), poly(propylene glycol), poly(ethylene terephthalate), poly(ethylene succinate), polyacetal, and polytetrahydrofuran, and the polyalkylene is selected from the group consisting of polyethylene, polypropylene and polybutadiene.

29. The composition of claim 28 further comprising a volatile organic solvent.
